(19) United States
(12) Patent Application Publication
(10) Pub. No.: US 2009/0017108 A1
(43) Pub. Date: Jan. 15, 2009

(54) Liposome compositions for treatment of hepatitis C

(76) Inventor: Alexander Yuzhakov, West Roxbury, MA (US)

Correspondence Address: Pearl Cohen Zedek Latzer, LLP, 1500 Broadway, 12th Floor, New York, NY, 10036, US

(21) Appl. No.: 12/216,830

(22) Filed: Jul. 11, 2008

Related U.S. Application Data
(60) Provisional application No. 60/959,172, filed on Jul. 11, 2007.

(51) Int. Cl. A61K 9/127; A61P 31/14; A61K 9/133; A61K 38/21

(52) U.S. Cl. 424/450; 424/85.7

(57) ABSTRACT

In the present invention compositions and methods are described for treating patients with a hepatitis C viral infection typically using interferon-alpha and/or ribavirin which are incorporated into liposomes, wherein the liposomes typically contain at least one carbohydrate moiety on the surface, such as a mono-, di-, oligo-, or polysaccharide moiety or any combination thereof, for targeting to hepatocytes. Other liposome surface modifications are provided to enhance effectiveness of the compound(s) therein.

### CROSS-REFERENCE TO RELATED APPLICATION

This application claims priority under 35 U.S.C. §119(e) to U.S. provisional patent application Ser. No. 60/959,172, filed Jul. 11, 2007, which is incorporated herein by reference in its entirety.

### BACKGROUND OF THE INVENTION

Infection with hepatitis C virus (HCV) is one of the leading causes of chronic liver disease throughout the world with an estimated human seroprevalence of 1% globally [Purcell, R. H., “Hepatitis C virus: Historical perspective and current concepts” FEMS Microbiology Reviews 14, pp. 181-192 (1994); Van der Poel, C. L., “Hepatitis C Virus. Epidemiology, Transmission and Prevention in Hepatitis C Virus. Current Studies in Hematology and Blood Transfusion, H. W. Reesink, Ed., (Basel: Karger), pp. 137-163 (1994)]. Four million individuals may be infected in the United States alone [Alter, M. J. and Mast, E. E., “The Epidemiology of Viral Hepatitis in the United States, Gastroenterol. Clin. North Am. 23, pp. 437-455 (1994)]. Chronic infection nearly always ensues after acute exposure to HCV, and chronically infected individuals develop cirrhosis and hepatocellular carcinoma at a dramatically elevated rate compared with the rate of disease in an uninfected population. [Kew, M. C., “Hepatitis C and Hepatocellular Carcinoma”, FEMS Microbiology Reviews, 14, pp. 211-220 (1994); Saito, I., et al. “Hepatitis C Virus Infection is Associated with the Development of Hepatocellular Carcinoma” Proc. Natl. Acad. Sci. USA 87, pp. 6547-6549 (1990)].

Interferons are a subclass of cytokines that exhibit both antiviral and antiproliferative activity. On the basis of biochemical and immunological properties, the naturally-occurring human interferons are grouped into three classes: interferon-alpha (leukocyte), interferon-beta (fibroblast) and interferon-gamma (immune). At least fourteen alpha interferons (grouped into subtypes A through H) having distinct amino acid sequences have been identified by isolating and sequencing DNA encoding these polypeptides. Alpha interferons have received considerable attention as potential therapeutic agents due to their antiviral and antitumor growth inhibition.

HCV is one of several clinical indications for which interferons have been approved by the Food and Drug Administration, and IFN-alpha is currently licensed for use in chronic HCV [Hoofnagle et al., Interferon: Principles and Medical Applications, 1st Edition, Chap. 31, pgs 433-462, 1992]. The types of responses that occur during IFN-alpha therapy can be characterized as: (1) a sustained complete response (“durable”), where patients serum ALT values begin to fall within the first months of treatment, are often normal by two to three months, and remain normal even after therapy is stopped. (these patients may also become negative for serum HCV RNA); (2) a transient complete response followed by relapse when therapy is stopped (“relapse”); (3) a partial or transient response, where patients serum ALT values decrease but do not become normal or become normal transiently and then rise despite continuation of interferon therapy (“partial response”); and (4) no response, where patients serum ALT activities remain elevated during interferon treatment (“non-response”).

The most frequently reported side-effects for IFN treatment are flu-like syndrome (68%), headache (40%), fatigue (39%), myalgia (29%) and local reaction at the injection site (29%). These symptoms typically occurred within the first month of therapy and subsided during the course of therapy. Neutropenia and thrombocytopenia induced by pegylated interferon alpha-2b increased the risk of infections and bleeding complications [Zonneveld, M., et al. “The Safety of Pegylated Interferon Alpha-2b in the Treatment of Chronic Hepatitis B: Predictive Factors for Dose Reduction and Treatment Discontinuation” Alimentary Pharmacology & Therapeutics (2005) 21, N9]. Also, significant amount of patients with IFN treatment have of anemia, depression and decrease in neutrophil count and a flu-like syndrome [Tripi, S., et al., “Interferon-alpha Alone versus Interferon-alpha plus Ribavirin in Patients with Chronic Hepatitis C Not Responding to Previous Interferon-alpha Treatment” (2000) Biodrugs. 13 pp. 299-304]. Long-term therapy of IFN induced pulmonary arterial Hypertension [Jochmann, N., et al. “Long-term therapy of interferon-alpha induced pulmonary arterial hypertension with different PDE-5 inhibitors: a case report” Cardiovascular Ultrasound (2005), 3 pp. 26]. Among the vascular complications reported for IFN are retinopathy [Kawano, T., et al., “Retinal complications during interferon therapy for chronic hepatitis” C. Am J Gastroenterol (1996), 91 pp. 309-3131, cutaneous vasculitis [Sangster, G., et al., “Cutaneous vasculitis associated with interferon.” Eur J Cancer Clin Oncol (1983) 19 pp. 1647-1649], gangrene requiring amputation and biopsy proven pulmonary vasculitis [Al-Zahrani, H., et al., “Vascular events associated with alpha interferon therapy. Leuk Lymphoma (2003), 44 pp. 471-475].

### SUMMARY OF THE INVENTION

In a first embodiment, a composition is provided for treating a hepatitis C viral infection among other diseases, said composition comprising liposomes comprising interferon-alpha, ribavirin, or the combination thereof. On one embodiment, the liposomes comprise interferon-alpha. In another embodiment the liposomes comprise interferon-alpha as the only antiviral agent. In another embodiment, the liposomes comprise interferon-alpha and ribavirin. In a further embodiment, the liposomes are unilamellar liposomes, multilamellar liposomes, multivesicular liposomes or any combination thereof. In a further embodiment, the liposomes are unilamellar liposomes. In a further embodiment, the liposomes are multilamellar liposomes. In another embodiment the liposomes are multivesicular liposomes. In yet a further embodiment, the liposomes have a diameter of from about 5 nanometers to about 25 micrometers. In another embodiment, the diameter is from about 5 nanometers to about 1 micrometer. In another embodiment, the diameter is from about 5 nanometers to about 100 nanometers.

In another embodiment, the liposomes comprise a mixture of lipids. In yet another embodiment, the liposomes comprise cholesterol. In another embodiment, the liposomes comprise phosphatidylserine. In another embodiment the liposomes comprise phosphatidylethanolamine. In another embodiment, the liposomes do not comprise phosphatidylserine.

In another embodiment, the liposomes comprise phosphatidylcholine, phosphatidylethanolamine and cholesterol. In another embodiment the liposomes do not comprise phosphatidylserine. In another embodiment, the ratio of phosphatidylcholine to cholesterol to phosphatidylethanolamine is about 20-30 parts to about 10-20 parts to one part. In another embodiment, the ratio is about 20-25 parts to about 15-20 parts to one part. In another embodiment the ratio is about 23 to about 16 to about one.

In yet another embodiment, the liposomes comprise at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In one embodiment, the at least one carbohydrate is a monosaccharide, disaccharide, oligosaccharide, or polysaccharide. By way of non-limiting example, the monosaccharide is mannose, galactose or fucose. In another non-limiting example, the disaccharide is lactose. In another embodiment the oligosaccharide is raffinose. In another embodiment the polysaccharide is pullulan.

In another embodiment, the liposomes comprise at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In one embodiment the glycoprotein is hepatitis B surface protein or human serum albumin. In another embodiment, the glycoprotein is asialylated, such as but not limited to asialofetuin.

In another embodiment, the liposomes comprise at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In a non-limiting example, the glycolipid is a ganglioside.

In another embodiment, the liposomes comprise at least one ceramide on the exterior surface thereof, on the interior surface thereof, or on both the exterior and interior surfaces thereof. In another embodiment, the liposomes comprise at least one ceramide on the exterior surface thereof. In another embodiment, the liposomes comprise at least one ceramide on the interior surface thereof. In another embodiment, the liposomes comprise at least one ceramide on the exterior surface thereof and on the interior surface thereof. In a non-limiting example, the ceramide is lactosyl-ceramide.

In yet a further embodiment, the liposomes are cationic. In a still further embodiment, the liposomes are anionic. In other embodiments the liposomes have both a positive and a negative charge.

In a further embodiment of the liposomes described above, the interferon-alpha is pegylated interferon-alpha, glycosylated interferon-alpha, a hydrophobic interferon-alpha, or a heparin-modified interferon-alpha.

In still yet a further embodiment, the liposomes in the composition described herein above are targeted to hepatocytes. In one embodiment, the targeting comprises liposomes that are unilamellar. In another embodiment the targeting comprises liposomes with a diameter less than about 5 micrometers. In another embodiment, the targeting comprises liposomes with a ratio of lipids as described above. In another embodiment, the molar ratio of phosphatidylcholine to cholesterol to phosphatidylethanolamine is about 20-30 parts to about 10-20 parts to one part. In another embodiment, the molar ratio is about 20-25 parts to about 15-20 parts to one part. In another embodiment the molar ratio is about 23 to about 16 to about one. In another embodiment, the targeting comprises a sugar, lipid or protein on the surface.

In one embodiment, the liposomes are targeted by means of at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In a further embodiment, the at least one carbohydrate is a monosaccharide, disaccharide, oligosaccharide, or polysaccharide, such as but not limited to mannose, galactose, fucose, or lactose. In another embodiment the oligosaccharide is raffinose. In another embodiment the polysaccharide is pullulan.

In another embodiment, the liposomes are targeted by means of at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In a further embodiment, the glycoprotein is asialylated, such as but not limited to asialofetuin. In another embodiment, the glycoprotein is hepatitis B surface protein or human serum albumin.

In another embodiment, the liposomes are targeted by means of at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In one embodiment, the glycolipid is a ganglioside.

In another embodiment, the liposomes are targeted by means of at least one ceramide on the exterior surface thereof, on the interior surface thereof, or on both the exterior and interior surfaces thereof. In one embodiment, the ceramide is lactosyl-ceramide.

In other embodiments, the liposomes are targeted by means of cationic charges thereon or by anionic charges thereon.

In a further embodiment, a method is provided for treating a hepatitis C viral infection or other disease comprising administering to a subject in need thereof a therapeutically effective amount of a pharmaceutical composition comprising liposomes comprising interferon-alpha, ribavirin, or the combination thereof. In one embodiment the liposomes comprise interferon-alpha. In another embodiment, the liposomes comprise interferon-alpha as the only antiviral agent. In another embodiment, the liposomes comprise interferon-alpha and ribavirin. In a further embodiment, the liposome composition is administered parenterally. In another embodiment, the liposome composition is administered between about once monthly to about once daily. In another embodiment, the liposome composition is administered about once weekly.

In a further embodiment, the liposomes useful for the methods described above are unilamellar liposomes, multilamellar liposomes, multivesicular liposomes or any combination thereof. In a further embodiment, the liposomes useful for the methods described above are unilamellar liposomes. In a further embodiment, the liposomes useful for the methods described above are multilamellar liposomes. In a further embodiment, the liposomes useful for the methods described above are multivesicular liposomes. In yet a further embodiment, the liposomes have a diameter of from about 5 nanometers to about 25 micrometers. In another embodiment, the diameter is from about 5 nanometers to about 1 micrometer. In another embodiment, the diameter is from about 5 nanometers to about 100 nanometers.

In another embodiment, the liposomes comprise phosphatidylcholine, phosphatidylethanolamine and cholesterol. In another embodiment the liposomes do not comprise phosphatidylserine. In another embodiment, the molar ratio of phosphatidylcholine to cholesterol to phosphatidylethanolamine is about 20-30 parts to about 10-20 parts to one part. In another embodiment, the molar ratio is about 20-25 parts to about 15-20 parts to one part. In another embodiment the molar ratio is about 23 to about 16 to about one.

In yet another embodiment, the liposomes useful for the methods described above comprise at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In one embodiment, the at least one carbohydrate is a monosaccharide, disaccharide, oligosaccharide, or polysaccharide. By way of non-limiting example, the monosaccharide is mannose, galactose or fucose. In another non-limiting example, the disaccharide is lactose. In another embodiment the oligosaccharide is raffinose. In another embodiment the polysaccharide is pullulan.

In another embodiment, the liposomes useful for the methods described above comprise at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In one embodiment the glycoprotein is hepatitis B surface protein or human serum albumin. In another embodiment, the glycoprotein is asialylated, such as but not limited to asialofetuin.

In another embodiment, the liposomes useful for the methods described above comprise at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In a non-limiting example, the glycolipid is a ganglioside.

In another embodiment, the liposomes useful for the methods described above comprise at least one ceramide on the exterior surface thereof, on the interior surface thereof, or on both the exterior and interior surfaces thereof. In a non-limiting example, the ceramide is lactosyl-ceramide.

In a further embodiment, in the liposomes useful for the methods described above, the interferon-alpha is pegylated interferon-alpha, glycosylated interferon-alpha, a hydrophobic interferon-alpha, or a heparin-modified interferon-alpha.

In yet a further embodiment, the liposomes useful for the methods described above are cationic. In a still further embodiment, the liposomes are anionic. In another embodiment, the liposomes have both a positive and a negative charge on the surface.

In another embodiment, a composition is provided for treating a hepatitis C viral infection among other diseases, comprising liposomes comprising interferon-alpha, ribavirin, or the combination thereof, in a carrier, wherein the liposomes are targeted to hepatocytes. In another embodiment, the liposomes comprise interferon-alpha and ribavirin. In a further embodiment, the liposomes are unilamellar liposomes, multilamellar liposomes, multivesicular liposomes or any combination thereof. In yet a further embodiment, the liposomes have a diameter of from about 5 nanometers to about 25 micrometers. In another embodiment, the diameter is from about 5 nanometers to about 1 micrometer. In another embodiment, the diameter is from about 5 nanometers to about 100 nanometers.

In another embodiment, the liposomes comprise a mixture of lipids. In yet another embodiment, the liposomes comprise cholesterol. In another embodiment, the liposomes comprise phosphatidylethanolamine. In another embodiment the liposomes do not comprise phosphatidylserine.

In another embodiment, the liposomes comprise phosphatidylcholine, phosphatidylethanolamine and cholesterol. In another embodiment the liposomes do not comprise phosphatidylserine. In another embodiment, the molar ratio of phosphatidylcholine to cholesterol to phosphatidylethanolamine is about 20-30 parts to about 10-20 parts to one part. In another embodiment, the molar ratio is about 20-25 parts to about 15-20 parts to one part. In another embodiment the molar ratio is about 23 to about 16 to about one.

In yet another embodiment, the liposomes comprise at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In one embodiment, the at least one carbohydrate is a monosaccharide, disaccharide, oligosaccharide, or polysaccharide. By way of non-limiting example, the monosaccharide is mannose, galactose or fucose. In another non-limiting example, the disaccharide is lactose. In another embodiment the oligosaccharide is raffinose. In another embodiment the polysaccharide is pullulan.

In another embodiment, the liposomes comprise at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In one embodiment the glycoprotein is hepatitis B surface protein or human serum albumin. In another embodiment, the glycoprotein is asialylated, such as but not limited to asialofetuin.

In another embodiment, the liposomes comprise at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In a non-limiting example, the glycolipid is a ganglioside.

In another embodiment, the liposomes comprise at least one ceramide on the exterior surface thereof, on the interior surface thereof, or on both the exterior and interior surfaces thereof. In a non-limiting example, the ceramide is lactosyl-ceramide.

In yet a further embodiment, the liposomes are cationic. In a still further embodiment, the liposomes are anionic.

In a further embodiment of the liposomes described above, the interferon-alpha is pegylated interferon-alpha, glycosylated interferon-alpha, a hydrophobic interferon-alpha, or a heparin-modified interferon-alpha.

In a further embodiment, a method is provided for treating a hepatitis C viral infection or other diseases comprising administering to a subject in need thereof a therapeutically effective amount of a pharmaceutical composition comprising liposomes comprising interferon-alpha, ribavirin, or the combination thereof, wherein the liposomes are targeted to hepatocytes. In another embodiment, the liposomes comprise interferon-alpha and ribavirin. In another embodiment the liposomes comprise interferon-alpha. In another embodiment the liposomes comprise interferon-alpha as the only antiviral agent. In a further embodiment, the liposome composition is administered parenterally. In another embodiment, the liposome composition is administered between about once monthly to about once daily. In another embodiment, the liposome composition is administered about once weekly.

In a further embodiment, the liposomes targeted to hepatocytes that are useful for the methods described above are unilamellar liposomes. In yet a further embodiment, the liposomes have a diameter of from about 5 nanometers to about 1 micrometer. In another embodiment, the diameter is from about 5 nanometers to about 100 nanometers.

In another embodiment, the liposomes targeted to hepatocytes and useful for the methods described above comprise a mixture of lipids. In yet another embodiment, the liposomes comprise cholesterol. In another embodiment, the liposomes comprise phosphatidylethanolamine. In another embodiment, the liposome does not comprise phosphatidylserine.

In another embodiment, the liposomes targeted to hepatocytes comprise phosphatidylcholine, phosphatidylethanolamine and cholesterol. In another embodiment the liposomes do not comprise phosphatidylserine. In another embodiment, the molar ratio of phosphatidylcholine to cholesterol to phosphatidylethanolamine is about 20-30 parts to about 10-20 parts to one part. In another embodiment, the molar ratio is about 20-25 parts to about 15-20 parts to one part. In another embodiment the molar ratio is about 23 to about 16 to about one.

In yet another embodiment, the liposomes targeted to hepatocytes and useful for the methods described above comprise at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In one embodiment, the at least one carbohydrate is a monosaccharide, disaccharide, oligosaccharide, or polysaccharide. By way of non-limiting example, the monosaccharide is mannose, galactose or fucose. In another non-limiting example, the disaccharide is lactose. In another embodiment the oligosaccharide is raffinose. In another embodiment the polysaccharide is pullulan.

In another embodiment, the liposomes targeted to hepatocytes and useful for the methods described above comprise at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In one embodiment the glycoprotein is hepatitis B surface protein or human serum albumin. In another embodiment, the glycoprotein is asialylated, such as but not limited to asialofetuin.

In another embodiment, the liposomes targeted to hepatocytes and useful for the methods described above comprise at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In a non-limiting example, the glycolipid is a ganglioside.

In another embodiment, the liposomes targeted to hepatocytes and useful for the methods described above comprise at least one ceramide on the exterior surface thereof, on the interior surface thereof, or on both the exterior and interior surfaces thereof. In a non-limiting example, the ceramide is lactosyl-ceramide.

In a further embodiment, in the liposomes targeted to hepatocytes and useful for the methods described above, the interferon-alpha is pegylated interferon-alpha, glycosylated interferon-alpha, a hydrophobic interferon-alpha, or a heparin-modified interferon-alpha.

In yet a further embodiment, the liposomes targeted to hepatocytes and useful for the methods described above are cationic. In a still further embodiment, the liposomes are anionic.

In still yet a further embodiment herein, the liposomes useful for the aforementioned methods are targeted to hepatocytes. In one embodiment, the liposomes are targeted by means of at least one carbohydrate moiety on the exterior surface thereof, on the interior surface thereof, or on both the exterior and interior surfaces thereof. In yet another embodiment, the liposomes comprise at least one carbohydrate moiety on the exterior surface thereof. In yet another embodiment, the liposomes comprise at least one carbohydrate moiety on the interior surface thereof. In yet another embodiment, the liposomes comprise at least one carbohydrate moiety on the exterior surface thereof and on the interior surface thereof. In a further embodiment, the at least one carbohydrate is a monosaccharide, disaccharide, oligosaccharide, or polysaccharide, such as but not limited to mannose, galactose, fucose, or lactose. In another embodiment the oligosaccharide is raffinose. In another embodiment the polysaccharide is pullulan.

In another embodiment of the aforementioned method, the liposomes are targeted by means of at least one glycoprotein on the exterior surface thereof, on the interior surface thereof, or on both the exterior and interior surfaces thereof. In another embodiment, the liposomes comprise at least one glycoprotein on the exterior surface thereof. In another embodiment, the liposomes comprise at least one glycoprotein on the interior surface thereof. In another embodiment, the liposomes comprise at least one glycoprotein on the exterior surface thereof and on the interior surface thereof. In a further embodiment, the glycoprotein is asialylated, such as but not limited to asialofetuin. In another embodiment, the glycoprotein is hepatitis B surface protein or human serum albumin.

In another embodiment, the liposomes are targeted by means of at least one glycolipid on the exterior surface thereof, on the interior surface thereof, or on both the exterior and interior surfaces thereof. In another embodiment, the liposomes comprise at least one glycolipid on the exterior surface thereof. In another embodiment, the liposomes comprise at least one glycolipid on the interior surface thereof. In another embodiment, the liposomes comprise at least one glycolipid on the exterior surface thereof and on the interior surface thereof. In one embodiment, the glycolipid is a ganglioside.

In another embodiment, the liposomes are targeted by means of at least one ceramide on the exterior surface thereof, on the interior surface thereof, or on both the exterior and interior surfaces thereof. In one embodiment, the ceramide is lactosyl-ceramide.

In other embodiments, the liposomes are targeted by means of cationic charges thereon or by anionic charges thereon.

In another embodiment, methods are provided for preparing liposomes that are targeted to hepatocytes.

### BRIEF DESCRIPTION OF THE FIGURE

FIG. 1 shows the distribution of liposomes embodied herein among various organs after intravenous administration to mice.

### DETAILED DESCRIPTION OF THE INVENTION

In the present invention compositions and methods are described for treating patients with hepatitis C viral infection among other diseases, typically using interferon-alpha and/or ribavirin which are incorporated into liposomes, wherein the liposomes typically contain at least one modification of a surface of the liposomes. Such modification is provided, by way of non-limiting examples, using at least one carbohydrate moiety on the surface, such as a mono-, di-, oligo-, or polysaccharide moiety or any combination thereof. In other embodiments, a glycoprotein or glycolipid is used, or other lipid components. This invention provides a means to significantly reduce the amount of drugs that are needed for patient treatment, and thereby protecting the heart, lungs, kidneys, and other major organs from adverse effects of the interferon and/or ribavirin, to reduce or completely eliminate adverse effects of the drug(s). The liposomes embodied here comprising interferon-alpha typically are targeted to hepatocytes.

Interferon alfa-2a (ROFERON-A; Hoffmann-La Roche), interferon alpha-2b (INTRON-A; Schering-Plough) and interferon alfacon-1 (INFERGEN; Intermune) are all approved in the United States for the treatment of adults with chronic hepatitis C as single agents. The recommended dose of interferons alfa-2b and alpha-2a for the treatment of chronic hepatitis C is 3,000,000 units three times a week, administered by subcutaneous or intramuscular injection. Treatment is administered for six months to two years. For interferon alfacon-1, the recommended dose is 9 mcg three times a week for first time treatment and 15 mcg three times a week for another six months for patients who do not respond or relapse. There are two preparations of pegylated interferon (peginterferon) alpha that have been studied in patients with hepatitis C: peginterferon alpha-2b (PEG-INTRON; Schering-Plough) and peginterferon alpha-2a (PEGASYS; Hoffmann-La Roche). The differences between these two preparations are subtle and most data suggest that they are equivalent with regards to efficacy and side effect profile. Peginterferon alphas differ from the older, unmodified interferon alphas in that a polyethylene glycol molecule is attached to the interferon molecule. As a result its elimination from the body is slowed and higher, more constant blood levels of interferon alpha are achieved with less frequent dosing. In contrast to unmodified interferon alpha, which must be injected three times a week to treat chronic hepatitis C, peginterferon alpha needs to be injected only once a week. With peginterferon alpha-2a alone, approximately 30% to 40% of patients achieve a sustained response to treatment for 24 to 48 weeks [Zeuzem S, Feinman S V, Rasenack J, Heathcote E J, Lai M Y, Gane E, O'Grady J, Reichen J, Diago M, Lin A, Hoffman J, and Brunda M J. “Peginterferon alfa-2a in patients with chronic hepatitis C” N Engl J. Med. (2000) 343, pp. 1666-72; Heathcote E J, Shiffman M L, Cooksley W G, Dusheiko G M, Lee S S, Balart L, Reindollar R, Reddy R K, Wright T L, Lin A, Hoffman J, De Pamphilis J. “Peginterferon alfa-2a in patients with chronic hepatitis C and cirrhosis” (2000) N Engl J. Med. 343 pp. 1673-80; Lee S S, Bain V G, Peltekian K, Krajden M, Yoshida E M, Deschenes M, Heathcote J, Bailey R J, Simonyi S, Sherman M; CANADIAN PEGASYS STUDY GROUP. “Treating chronic hepatitis C with pegylated interferon alfa-2a (40 KD) and ribavirin in clinical practice” Alignment Pharmacol Ther. (2006) 23 pp. 397-408].

Ribavirin is a synthetic nucleoside that has activity against a broad spectrum of viruses. In several studies, oral ribavirin was examined as a single agent for the treatment of adults with chronic hepatitis C. Although decreases in serum ALT activities were seen with treatment, the overall results of these studies were discouraging as sustained-responses were rarely achieved. The FDA did not approve ribavirin alone for hepatitis C. Because of its partial effectiveness, ribavirin was studied in subsequent trials in combination with interferon alpha. FDA approval of interferon alpha-2b plus ribavirin for the treatment of individuals with chronic hepatitis C who “relapsed” after previous interferon alpha therapy. Subjects in these trials received either injections of interferon alpha-2b at a dose of 3 million units three times a week and either oral ribavirin at a dose of 1.0 g to 1.2 g daily or a matched placebo for 24 weeks of treatment. Six months after treatment was discontinued, 45.7% of subjects who received interferon alpha-2b plus ribavirin had undetectable serum viral RNA as compared to 4.7% who received only interferon alpha-2b. Subsequent studies showed that the combination of interferon alpha-2b plus ribavirin is more effective in achieving a sustained response than interferon alpha-2b alone in the treatment of patients with chronic hepatitis C not previously treated with interferon. This led to FDA approval for this indication in December 1998. Results from three double-blind, placebo-controlled trials supporting this were published in 1998 [Reichard O, Norkrans G, Fryden A, Braconier J H, Sonnerborg A, Weiland O. “Randomised, double-blind, placebo-controlled trial of interferon alpha-2b with and without ribavirin for chronic hepatitis C” The Swedish Study Group. Lancet. (1998) 351 pp. 83-7; Poynard T, Marcellin P, Lee S S, Niederau C, Minuk G S, Ideo G, Bain V, Heathcote J, Zeuzem S, Trepo C, Albrecht J. “Randomised trial of interferon alpha2b plus ribavirin for 48 weeks or for 24 weeks versus interferon alpha2b plus placebo for 48 weeks for treatment of chronic infection with hepatitis C virus” International Hepatitis Interventional Therapy Group (IHIT) Lancet. (1998) 352 pp. 1426-32; McHutchison J G, Gordon S C, Schiff E R, Shiffman M L, Lee W M, Rustgi V K, Goodman Z D, Ling M H, Cort S, Albrecht J K. “Interferon alfa-2b alone or in combination with ribavirin as initial treatment for chronic hepatitis C.” Hepatitis Interventional Therapy Group. N Engl J Med. (1998) 339 pp. 1485-92].

Liposomes (vesicles) are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer). Liposomes smaller than about 200 nm usually only consist of one bilayer (unilamellar liposomes) but larger liposomes can contain concentric layers of lipid or several smaller liposomes can be formed inside large liposomes. These larger multicompartmented liposomes are known as multilamellar liposomes. Multivesicular liposomes are liposomes containing multiple non-concentric chambers within each liposome particle, resembling a “foam-like” matrix.

The bilayer is composed of two lipid monolayers having a hydrophobic “tail” region and a hydrophilic “head” region. The structure of the membrane bilayer is such that the hydrophobic (nonpolar) tails of the lipid monolayers orient towards the center of the bilayer while the hydrophilic heads orient towards the aqueous phase.

The original liposome preparation of Bangham et al. (J. Mol. Biol., 1965, 12 pp. 238-252) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. MLVs so formed may be used in the practice of the present invention.

Another class of multilamellar liposomes that may be used as the starting liposomes of this invention is that characterized as having substantially equal lamellar solute distribution. This class of liposomes is denominated as stable plurilamellar vesicles (SPLV) as defined in U.S. Pat. No. 4,522,803 to Lehk, et al., reverse phase evaporation vesicles (REV) as described in U.S. Pat. No. 4,235,871 to Papahadjopoulos et al., monophasic vesicles as described in U.S. Pat. No. 4,558,579 to Fountain, et al., and frozen and thawed multilamellar vesicles (FATMLV) wherein the vesicles are exposed to at least one freeze and thaw cycle; this procedure is described in Bally et al., PCT Publication No. 87/00043, Jan. 15, 1987, entitled “Multilamellar Liposomes Having Improved Trapping Efficiencies”; these references are incorporated herein by reference.

Unilamellar liposomes can be made using sonication or extrusion methods. Sonication typically produces small, unilamellar vesicles (SUV) with diameters in the range of 15-50 nm. The most common instrumentation for preparation of sonicated particles are bath and probe tip sonicators [Kasahara M. and Hinkle P. C. “Reconstitution of D-glucose transport catalyzed by a protein fraction from human erythrocytes in sonicated liposomes.” Proc Natl Acad Sci USA. 1976 February; 73(2): 396-400]. Extrusion is a technique in which a lipid suspension is forced through a polycarbonate filter with a defined pore size to yield particles having a diameter near the pore size of the filter used [Gallová J, Uhríková D, Islamov A, Kuklin A, Balgavý P. “Effect of cholesterol on the bilayer thickness in unilamellar extruded DLPC and DOPC liposomes: SANS contrast variation study.” Gen Physiol Biophys. 2004 March; 23(1):113-28].

Liposomes are comprised of lipids; the term lipid as used herein shall mean any suitable material resulting in a bilayer such that a hydrophobic portion of the lipid material orients toward the interior of the bilayer while a hydrophilic portion orients toward the aqueous phase. Exemplary but non-limiting lipids which can be used in the liposome formulations of the present invention are the phospholipids such as phosphatidylcholine (PC), phosphatidylglycerol (PG), phosphatidylethanolamine (PE) and phosphatidylserine (PS), and more particularly, dimyristoylphosphatidylcholine (DMPC) and dimyristoylphosphatidylglycerol (DMPG). Liposomes may be formed and vesiculated using DMPG, or DMPG mixed with DMPC in, for example, a 3:7 mole ratio, respectively. In certain embodiments herein, the liposomes do not contain phosphatidylserine.

During preparation of the liposomes, organic solvents may be used to suspend the lipids. Suitable organic solvents are those with intermediate polarities and dielectric properties, which solubilize the lipids, and include but are not limited to halogenated, aliphatic, cycloaliphatic, or aromatic-aliphatic hydrocarbons, such as benzene, chloroform, methylene chloride, or alcohols, such as methanol, ethanol, and solvent mixtures such as benzene:methanol (70:30). As a result, solutions (mixtures in which the lipids and other components are uniformly distributed throughout) containing the lipids are formed. Solvents are generally chosen on the basis of their biocompatability, low toxicity, and solubilization abilities.

The starting multilamellar liposomes and resulting unilamellar liposomes of the present invention may also contain lipid soluble bioactive agents. The vesicles contain bilayer-associated molecules such as proteins or peptides. Multilamellar liposomes have advantages to one-layer liposomes: the multiplicity of coats increases the effect of a reservoir and makes extremely prolonged releases of drugs possible.

Multivesicular liposomes are design for a depot delivery system to overcome the limitations of conventional therapies and have in vivo effectiveness for sustained delivery. Multivesicular liposomes can be prepared by the reverse phase evaporation method [Jain S. K., Jain, R. K., Chourasia, M. K., Jain, A. K., Chalasani, K. B. Soni, V. Jain A. “Design and Development of Multivesicular Liposomal Depot Delivery System for Controlled Systemic Delivery of Acyclovir Sodium” (2005) AAPS PharmSciTech 6, pp. 35-41]. Multivesicular liposomes are characterized by their sui-generis structure of multiple nonconcentric aqueous chambers surrounded by a network of lipoidal membranes. These systems can be differentiated from conventional liposomes (unilamellar and multilamellar liposomes) in 2 major aspects: size and composition. Multivesicular liposomes have a size range of 5 to 25 μm compared with unilamellar (up to 1 μm) and multilamellar (1-5 μm). In addition to the general chemical composition of conventional liposomes, multivesicular liposomes can contain a neutral lipid (triolein, tricaprylene, trilaureine, tributyrine, etc) as an integral component, which is responsible for its unique multivesicular structure. As multivesicular liposomes are composed of multiple nonconcentric aqueous chambers, they contain 95% water, therefore providing a unique carrier system for hydrophilic drugs including a variety of therapeutic proteins [Kim S. “Depofoam mediated drug delivery into cerebrospinal fluid” Methods Neuroscience. (1994) 21, pp. 118-131; Kim T, Kim J, Kim S. “Extended release formulation of morphine for subcutaneous administration” Cancer Chemother Pharmacol. (1993) 33, pp. 187-190].

As mentioned above, additional components provided in liposomes embodied herein can provide enhanced efficacy of the compositions herein. In one embodiment, a combination of cholesterol, phosphatidylcholine and/or phosphatidylethnolamine can be included in the liposome membrane to enhance activity. In another embodiment, the efficiency of the liposomes embodied herein is enhanced by the presence of saccharide moieties and other cell-type specific agents. Several different saccharolipids with the wide spectrum of carbohydrate moieties covalently attached to the head of the vesicle-forming lipids can be produced by a variety of methods. Water soluble carrier agents can also be incorporated into the vesicles of this invention by the procedure of Szoka [Szoka F. Jr., Papahadjopoulos D. “Procedure for preparation of liposomes with large internal aqueous space and high capture by reverse-phase evaporation” Proc Natl Acad Sci USA. (1978) 75 pp. 4194-4198]. This procedure is particularly useful for fragile water-soluble materials such as interferon.

Other liposome components useful to enhance effectiveness include glycoproteins and glycolipids. Such components, such as but no limited to ganglioside, ceramide, lactosyl-ceramide and human serum albumin can be incorporated into the liposomal components following methods as described above. In one embodiment, the glycoprotein is asialylated, such as asialofetuin. These examples are non-limiting.

Liposomes embodied here comprising interferon-alpha that are targeted to hepatocytes can be prepared as follows. Phospholipids can be purchased from any number of sources, such as Avanti Polar Lipids. Human interferon alpha (rhIFN-αA) can be obtained from Calbiochem. Other components such as cholesterol and buffers can be purchased from Sigma Chemical Co. To prepare lipids from stock solutions of egg phosphatidylcholine, cholesterol, and L-α-phosphatidylethanolamine in chloroform were mixed in a molar ratio of 23:16:1, dried under reduced nitrogen pressure, dissolved in cyclohexane, and lyophilized. The liposomes then were hydrated in buffer (10 mM Hepes, 135 mM NaCl, pH 6.7) in the presence of 2.8 μg/ml or 500K units/ml IFN and sized by repeated extrusion (4 times) through polycarbonate filters with a pore size of 100 nm (Costar) at 25° C. and 5 times through 50 nm at 50° C. As will be seen in the examples below, such liposomes preferentially delivered the interferon-alpha to liver.

In various animal species and humans, liposomes are generally cleared rapidly by the cells of the mononuclear phagocyte system, especially macrophages in the spleen and Kupffer cells in the liver. When liposomes are small enough to be able to pass the fenestrae in the endothelial cell lining, they also will have access to hepatocytes. In another embodiment herein, the liposomes embodied herein are targeted to hepatocytes, i.e., are modified in order to be attracted to, bind to, or otherwise localize at hepatocytes, and typically taken up by and/or release their contents thereby or therein. Various means are described herein for achieving the aforementioned targeting, and non-limiting examples are provided herein. For example, coupling of polyaconitylated human serum albumin to liposomes leads to a 17-fold increase in liver uptake, as compared with control liposomes, whereas spleen uptake is not affected [Kamps J. A. A. M., Morselt H. W. M., Swart P. J., Meijer D. K. F., Scherphof G. L. “Massive targeting of liposomes, surface-modified with anionized albumins, to hepatic endothelial cells” Proc. Natl. Acad. Sci. USA 94, pp. 11681-11685]. Moreover, the various sugar modifications described herein are also useful as targeting means. In one embodiment, cholesterol or phosphatidylserine can be included in the liposome membrane to enhance targeting. In another embodiment, the efficiency of the hepatocyte targeting systems is provided by the presence of saccharide moieties and other cell-type specific agents. In one embodiment an oligosaccharide can be included, such as but not limited to raffinose. In another embodiment a polysaccharide can be included, such as but not limited to pullulan. Sugar-like structures are useful for efficient binding to the galactose receptor of hepatocytes [Stults, N. L. and Lee, Y. C. “Enhancement of galactose/N-acetylgalactosamine receptor activity on the surface of freshly isolated rat hepatocytes: Evidence for masking of receptor sites by inhibitors derived from collagenase preparations” (1986) Proc. Natl. Acad. Sci. USA Vol. 83, pp. 7775-7779]. Several different saccharolipids with the wide spectrum of carbohydrate moieties covalently attached to the head of the vesicle-forming lipids can be produced by a variety of methods [Remy J. S., Kichler A., Mordvinov V., Schuber F., Behr J. P. “Targeted gene transfer into hepatoma cells with lipopolyamine-condensed DNA particles presenting galactose ligands: A stage toward artificial viruses” (1995) Proc. Natl. Acad. Sci. USA Vol. 92, pp. 1744-1748]. Water soluble carrier agents can also be incorporated into the vesicles of this invention by the procedure of Szoka [Szoka F. Jr., Papahadjopoulos D. “Procedure for preparation of liposomes with large internal aqueous space and high capture by reverse-phase evaporation” Proc Natl Acad Sci USA. (1978) 75 pp. 4194-4198]. This procedure is particularly useful for fragile water-soluble materials such as interferon.

Other liposome components useful to enhance targeting include glycoproteins and glycolipids. Such components, such as but no limited to ganglioside, ceramide, lactosyl-ceramide and human serum albumin can be incorporated into the liposomal components following methods as described above. In one embodiment, the glycoprotein is asialylated, such as asialofetuin. These examples are non-limiting.

Cationic liposomes are widely used for gene transfection in vitro and have been tested for gene delivery to the liver. Templeton et al. showed that cationic liposomes generated from 1,2-bis(dioleoyloxy)-3-(trimethylammonio) propane (DOTAP) and cholesterol (Chol) were effective gene transfer agents in vivo. [Templeton N S, Lasic D D, Frederik P M, Strey H H, Roberts D D, Pavlakis G N. “Improved DNA: liposome complexes for increased systemic delivery and gene expression.” Nat Biotechnol. (1997) 15 pp. 647-652]. The liposomes generated from poly(cationic lipid) and cholesterol have low cytotoxicity, are serum resistant, and display a transfection efficiency in vitro similar to commercially available cationic liposomes [Liu L, Zem M A, Lizarzaburu M E, Nantz M H, Wu J. “Poly(cationic lipid)-mediated in vivo gene delivery to mouse liver.” (2003) Gene Therapy (2003) 10, 180-187].

Among the various diseases and conditions amendable to therapy using the compositions described herein, and for use on humans as well as other vertebrate species, interferon α-2b is used for the treatment of hairy cell leukemia, malignant melanoma, condylomata acuminata, AIDS-related Kaposi's sarcoma, chronic hepatitis C, and chronic hepatitis B. Ribavirin in conjunction with interferon is approved for the treatment of chronic hepatitis C. Other interferons are also amenable to the methods described herein. Interferon beta-1b (Betaseron) and interferon beta-1a (Avonex) are approved for treatment of multiple sclerosis. Interferon alfa-n3 (Alferon-N) is prescribed for treatment of genital and perianal warts caused by human papillomavirus (HPV). Interferon gamma-1B (Actimmune) is used for treatment of chronic granulomatous disease, and severe, malignant osteopetrosis. The foregoing examples are non-limiting.

In addition to the forms of interferons described above, the interferon used in the embodiments herein may be modified. Modifications such as, but not limited to, pegylation, glycosylation, hydrophobic modification, and conjugation to heparin are further means to increase the effectiveness of the interferon. Pegylation is mentioned above.

The liposomes of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into or around areas infected by the virus. Typically, liposomes embodied herein are administered parenterally. In some embodiments, a depot injection is administered. In a typical embodiment, the liposome composition of the invention is administered intravenously.

The dosage amounts are based on the effective inhibitory concentrations observed in anti-viral studies. The preferred route will vary with the (1) condition and age of the recipient, (2) the virus being treated (3) nature of the infection and (4) desired blood levels. One of skill in the art can readily determine the appropriate dose and route based on considering these clinical determinations. Parenteral treatment by intravenous, subcutaneous, or intramuscular application of the liposomes of the present invention formulated with an appropriate carrier, other antiviral agents or compounds or diluents to facilitate application will be the preferred method of administering the compounds to warm blooded animals.

Assessment of the targeting of the liposomes embodied here as well as efficacy in the treatment of hepatitis C infection and other diseases can be readily determined in animal models prior, and activity in such models is predictive of potential human therapeutic benefit. By way of non-limiting examples, various models are described in [Kremsdorf D, Brezillon N., 2007, “New animal models for hepatitis C viral infection and pathogenesis studies” World J Gastroenterol. 13 pp. 2427-35]. For example, fetal rats are tolerized by an intraperitoneal injection of Huh7 cells into pregnant females at the 17th day of gestation. Twenty-four hours after birth, the rats are intrasplenically transplanted with the same hepatoma cell line which will represent around 6% of total hepatocytes after 14 days of development. Nearly 30% of the transplanted human hepatocytes are positive for HCV core protein after inoculation with HCV (genotype 1) positive human serum. For example, Wu et al. (A novel immunocompetent rat model of HCV infection and hepatitis, Gastroenterology 2005; 128(5):1416-23), find Huh 7 cells in the liver, and HCV viral replication was detected by the presence of negative strand HCV RNA. HCV levels in serum were measured at 11,000 copies/mL at week 4, peaked at 22,500 copies/mL by week 12. In tolerized, transplanted, inoculated rats, but not controls, serum alanine aminotransferase (ALT) values increased to 60 IU/L by week 4 and reached a peak of approximately 120 IU/L by week 13. Histology showed foci of mononuclear infiltrates in portal and central regions. These measures of HCV infection are used to evaluate the effectiveness of the liposomes embodied herein.

This, in certain embodiments herein, liposomes comprising interferon-alpha are provided. In another embodiment, liposomes comprise interferon-alpha as the only antiviral or biologically active agent. In another embodiment, liposomes comprising interferon-alpha that are unilamellar liposomes are provided. In another embodiment. The unilamellar liposomes comprise interferon-alpha as the only antiviral or biologically active agent. In another embodiment, the liposomes do not comprise phosphatidylserine as a lipid component. In another embodiment, unilamellar liposomes comprising interferon-alpha that do not comprise phosphatidylserine are provided. In another embodiment, unilamellar liposomes comprising interferon-alpha as the only antiviral or biologically active agent are provided, which liposomes do not comprise phosphatidylserine. Any of the foregoing liposome embodiments can also be smaller than 5 micrometers in size, such as, in certain embodiments, around 5 nanometers to around 1 micrometer. In other embodiments, the unilamellar liposomes are from about 5 to 250 nanometers in diameter, and in other embodiments, around 50 to 150 nanometers. In other embodiments the liposomes are around 100 nanometers in diameter. Any of the foregoing liposome embodiments can also comprise polyaconitylated human serum albumin on the outer surface. Any of the foregoing liposome embodiments can comprise a glycolipid (e.g., a ganglioside, ceramide, or lactosyl-ceramide) or glycoprotein (e.g., asialofetuin or hepatitis B surface protein) as described elsewhere herein. Any of the foregoing liposome embodiments can comprise a carbohydrate on the surface thereof, such as a monosaccharide (e.g., mannose, galactose, fucose); or a disaccharide (e.g., lactose). In another embodiment an oligosaccharide can be included, such as but not limited to raffinose. In another embodiment a polysaccharide can be use, such as but not limited to pullulan. Any of the foregoing liposome embodiments can comprise one or more hepatocyte targeting agents.

### EXAMPLES

### Preparation of Unilamellar Liposomes

Unilamellar liposomes of about 100 nm average diameter are prepared by the extrusion method [Mayer L D, Hope M J, Cullis P R. “Vesicles of variable sizes produced by a rapid extrusion procedure” Biochim Biophys Acta. (1986) 858 pp. 161-8] employing a laboratory extruder (LiposoFast-Pneumatic, Avestin Inc.) [MacDonald R C, MacDonald R I, Menco B P, Takeshita K, Subbarao N K, Hu L R. “Small-volume extrusion apparatus for preparation of large, unilamellar vesicles” Biochim Biophys Acta. (1991) 1061 pp. 297-303; Pantos A, Tsiourvas D, Paleos C M, Nounesis G. “Enhanced drug transport from unilamellar to multilamellar liposomes induced by molecular recognition of their lipid membranes” Langmuir. (2005) 21 pp. 6696-702]. In a typical experiment for preparing a 2 mL dispersion of liposomes, 0.038 mmol of soybean hydrogenated phosphatidylcholine (PC), 0.019 mmol of cholesterol (CHOL) (molar ratio PC:CHOL)2:1), and 0.002 mmol of ODPG (molar ratio PC:ODPG 19:1) are dissolved in chloroform/methanol (2:1, v/v) for the formation of lipid films. For the preparation of unilamellar liposomes containing interferon, ribavirin, or their combination, 1 mg of these agents is also added to this solution. The lipid film is hydrated with 2 mL of phosphate-buffered saline, PBS (50 mM phosphate buffer, pH 7.4, 300 mM NaCl), and the sample is vortexed for 10 min at about 60° C. The suspension obtained is extruded through two stacked polycarbonate filters of 100 nm pore size. Typically about ten, but up to twenty five cycles, are applied at 60° C. An analogous procedure is followed for the preparation of the saccharide-liposomes. In this case, in addition to phosphatidylcholine and cholesterol, saccharide-modified lipids are added at a 10% molar percentage relative to other lipids.

In a typical experiment, a dispersion of 2 mL of unilamellar liposomes in citrate buffer (300 mM, pH 4) is filtered through a Sephadex G-50 column using PBS as an eluent to exchange the external citrate buffer and establish a transmembrane pH gradient. Free components including unencapsulated interferon are removed during this step.

The unilamellar liposomes are typically around 5 nanometers to around 1 micrometer in diameter. By varying conditions of preparation, the unilamellar liposomes can me made from about 5 to 250 nanometers in diameter, or around 50 to 150 nanometers.

### Multilamellar Liposomes

Large multilamellar liposomes of 400 nm diameter are prepared by the extrusion method. In a typical experiment for preparing a 2 mL dispersion of liposomes, 0.038 mmol of phosphatidylcholine, 0.019 mmol of cholesterol (molar ratio 2:1), and 0.002 mmol (1.0 of DHP (molar ratio PC:DHP) 19:1) are dissolved in chloroform/methanol solution (2:1, v/v) for the formation of lipid films. The film is hydrated with 2 mL of PBS comprising interferon-alpha and optional other components, and the sample is vortexed for 10 min at 65° C. The suspension obtained is extruded through two stacked polycarbonate filters of 400 nm pore size; only three cycles were applied at 65° C. to obtain multilamellar liposomes [New, R. R. C. In Liposomes: A practical approach; Rickwood, D., Hames, B. D., Eds.; IRL Press: Oxford, U.K., 1990; pp 55-56]. After centrifugation at 12.000 g for 30 min, the multilamellar liposomes precipitated as a pellet while unilamellar liposomes remained dispersed in the supernatant layer. The pellet is washed two times with PBS and finally resuspended in 2 mL of PBS. Multilamellar liposomes are typically about 1 to about 5 micrometers in diameter.

### Cationic Liposomes

Cationic Liposomes are prepared as follows. The glycolipids are prepared by conjugation of mono-, di-, poly-, and oligo-saccharides with dipalmitoylphosphatidylcholine (DPPE) by reductive amination as described before [Shimizu Y, Yamakami K, Gomi T, Nakata M, Asanuma H, Tadakuma T, Kojima N. “Protection against Leishmania major infection by oligomannosecoated liposomes” (2003) Bioorg Med Chem 11 pp. 1191-5]. Poly(cationic lipid) and PCL-Chol, DOTAP-Chol, DOTAP-DOPE are generated in a molar ratio of 3:1 according to a method described previously [Wu J, Lizarzaburu M E, Kurth M J, Liu L, Wege H, Zern M A, Nantz M H. “Cationic lipid polymerization as a novel approach for constructing new DNA delivery agents” Bioconj Chem (2001) 12 pp. 251-257] and their size is measured before mixing with interferon alpha or ribavirin, or its combination (drugs). The size distribution of three formulations of cationic liposomes before mixing with drugs is between 200 and 300 nm. The mixture of cationic liposomes with the drugs is performed prior to use in a charge ratio of 1:1 according to a described method [Templeton N S, Lasic D D, Frederik P M, Strey H H, Roberts D D, Pavlakis G N. “Improved DNA:liposome complexes for increased systemic delivery and gene expression” Nat Biotechnol (1997) 15 pp. 647-652]. In brief, interferon solution at a concentration of 1 mg/ml is added dropwise to the liposome suspension and mixed by pipeting. The complexes can be kept at room temperature until injection.

### Multivesicular Liposomes

Multivesicular liposomes containing drug are manufactured by a two-step, water-in-oil-in-water double-emulsification process. The first emulsification process combines an aqueous phase containing interferon to be encapsulated with an organic phase containing lipids in solvent, such as chloroform or methylene chloride. The lipid solution used contains one or two phosphatidylcholines (typically monounsaturated), a phosphatidylglycerol, cholesterol, and one or two triglycerides. The triglyceride component of the lipids imparts the multivesicular structure to the multivesicular particle, without which the resulting particles would resemble conventional liposomes. The first emulsion is dispersed and mixed with a second aqueous solution to form a second, water-in-oil-in-water, emulsion. The second emulsion is flushed with nitrogen to remove the solvent, at which time numerous aqueous chambers (submicron- to micron sized), separated by lipid membranes, come together to form the closely packed multivesicular particle [Kim S, Turker M S, Chi E Y, et al. “Preparation of multivesicular liposomes” Biochim Biophys Acta (1983) 728 pp. 339-48]. This complex structure of the multivesicular particle also results in reasonable stability of formulation during storage, and control over the drug-release rate. Multivesicular liposomes are typically about 5 to about 25 micrometers in diameter.

### Derivatized Liposomes

Human serum albumin (HSA) derivatized liposome preparation: Lipids from stock solutions of egg phosphatidylcholine, cholesterol, and maleimido-4-(p-phenylbutyryl)-phosphatidylethanolamine in chloroformymethanol (9:1) are mixed in a molar ratio of 23:16:1, dried under reduced nitrogen pressure, dissolved in cyclohexane, and lyophilized. Preparation of HSA in which an average of 53 of the 60 available amino groups are derivatized with cis-aconitic anhydride and coupling of the protein to the liposomes [Kamps J. A. A. M., Morselt H. W. M., Swart P. J., Meijer D. K. F., Scherphof G. L. “Massive targeting of liposomes, surface-modified with anionized albumins, to hepatic endothelial cells” Proc. Natl. Acad. Sci. USA 94, pp. 11681-11685].

Preparation of Pegyated Interferon. Pegyated interferon is prepared as described in above, and additionally as described [Peg-interferon conjugates U.S. Pat. No. 5,382,657].

### Hepatocyte Targeted Liposomes

Phospholipids were obtained from Avanti Polar Lipids, human interferon (rhIFN-αA) was from Calbiochem, cholesterol, buffers, and other reagents were from Sigma Chemical Co.

Liposomes preparation: Lipids from stock solutions of egg phosphatidylcholine, cholesterol, and L-α-Phosphatidylethanolamine in chloroform were mixed in a molar ratio of 23:16:1, dried under reduced nitrogen pressure, dissolved in cyclohexane, and lyophilized. The liposomes then were hydrated in buffer [10 mM Hepes, 135 mM NaCl, pH 6.7] in the presence of 2.8 μg/ml or 500K units/ml IFN and sized by repeated extrusion (4 times) through polycarbonate filters with a pore size of 100 nm (Costar) at 25° C. and 5 times through 50 nm at 50° C.

Tissue Distribution Studies: For determination of interferon tissue distribution, two groups with 5-6 animals (mice) per group were designed. In the first group 360 ng or 100K units of IFN in 0.1 ml volume per animal (25 g weight) was administrated in tail vein under pentobarbital anesthesia. In the second group the same amount of liposomal capsulated interferon-alpha were injected under the same conditions. After 1.5 hours animals were sacrificed, blood and tissue samples were collected. The amount of interferon in organs was calculated based on data from ELISA kit (RnD Systems).

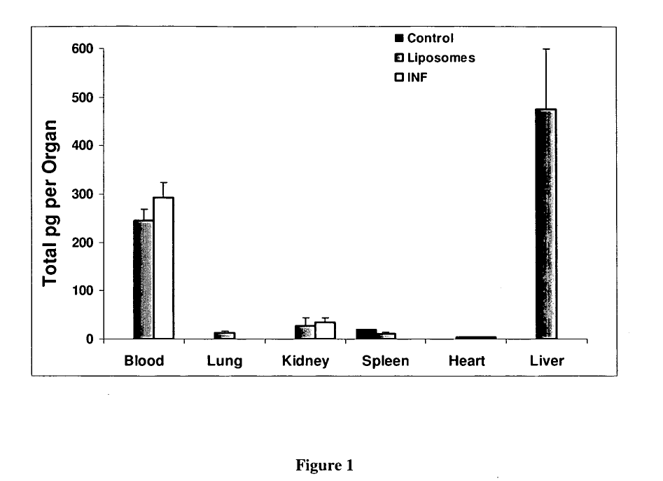

As shown in FIG. 1, interferon-alpha administered in the liposomes prepared as described above was preferentially taken up in the liver where the liposomes delivered the interferon-alpha.

### Assessment of Biological Activity

Rat hepatitis C (HCV) model: The fetal rats are tolerized by an intraperitoneal injection of Huh7 cells into pregnant females at the 17th d of gestation. Twenty-four hours after birth, the rats are intrasplenically transplanted with the same hepatoma cell line which will represent around 6% of total hepatocytes after 14 d of development. Nearly 30% of the transplanted human hepatocytes are positive for HCV core protein after inoculation with HCV (genotype 1) positive human serum. In addition, HCV gene expression, viral replication, plasma viral load, and development biochemical and histologic evidence of hepatitis occur in the model, including abnormal liver function (ALT elevation).

A liposome formulation comprising alpha-interferon in unilamellar liposomes is prepared as described above. In another experiment the same liposomes containing fucose modifications on the exterior surfaces are prepared. A pharmaceutical composition comprising the liposomes in a carrier is administered weekly to the HCV rat model. The equivalent 360 ng or 100,000 units of interferon-alpha in 0.1 mL is administered IV via tail vein. A beneficial effect on the pathology of the disease in this model is observed with regard to reduced viral replication and liver dysfunction.

What is claimed is:

1. A composition for treating a hepatitis C viral infection comprising liposomes comprising interferon-alpha, ribavirin, or the combination thereof, in a carrier.

2. The composition of claim 1 wherein the liposomes are unilamellar liposomes, multilamellar liposomes, concentric liposomes or the combination thereof.

3. The composition of claim 1 wherein the liposomes have a diameter of from about 5 nanometers to about 25 micrometers.

4. The composition of claim 3 wherein the diameter is from about 5 nanometers to about 1 micrometer.

5. The composition of claim 4 wherein the diameter is about 5 nanometers to about 100 nanometers.

6. The composition of claim 1 wherein the liposomes do not comprise phosphatidylserine.

7. The composition of claim 1, wherein said liposomes comprise at least one carbohydrate moiety, glycolipid moiety, glycoprotein moiety or asialylated glycoprotein moiety on the exterior surface thereof, on the interior surface thereof, or on both the exterior and interior surfaces thereof.

8. The composition of claim 7 wherein the at least one carbohydrate is a monosaccharide, disaccharide, oligosaccharide, or polysaccharide.

9. The composition of claim 7 wherein the at least one carbohydrate is mannose, galactose, fucose, lactose, raffinose or pullulan.

10. The composition of claim 7 wherein the asialylated glycoprotein is asialofetuin.

11. The composition of claim 7 wherein the glycoprotein is hepatitis B surface protein or human serum albumin.

12. The composition of claim 7 wherein the glycolipid is a ganglioside, a ceramide, or lactosyl-ceramide.

13. The composition of claim 1 wherein said interferon-alpha is pegylated interferon-alpha, glycosylated interferon-alpha, a hydrophobic interferon-alpha, or a heparin-modified interferon-alpha.

14. The composition of claim 1 wherein said liposomes are cationic, anionic, or have both a positive and a negative charge on the surface.

15. A method of treating a hepatitis C viral infection comprising administering to a subject in need thereof a therapeutically effective amount of a pharmaceutical composition comprising the liposomes of claim 1.

16. The method of claim 15 wherein the liposome composition is administered parenterally.

17. The method of claim 15 wherein the liposome composition is administered between about once monthly to about once daily.

18. The method of claim 17 wherein the liposome composition is administered about once weekly.

19. A method of targeting interferon-alpha therapy to hepatocytes for the treatment of a hepatitis C viral infection comprising administering to a subject in need thereof a therapeutically effective amount of a pharmaceutical composition comprising liposomes of claim 1.

20. A composition targeted to hepatocytes for treating a hepatitis C viral infection comprising liposomes of claim 1.
